(19) United States
(12) Patent Application Publication
(10) Pub. No.: US 2013/0211171 A1
(43) Pub. Date: Aug. 15, 2013

(54) METHOD FOR REMOVING ACID GASES FROM HYDROCARBON-COMPRISING FLUIDS

(71) Applicant: BASF SE, (US)

(72) Inventors: Christian Riemann, Altrip (DE); Gerd Modes, Calgary (CA)

(73) Assignee: BASF SE, Ludwigshafen (DE)

(21) Appl. No.: 13/765,840

(22) Filed: Feb. 13, 2013

Related U.S. Application Data
(60) Provisional application No. 61/598,402, filed on Feb. 14, 2012.

(51) Int. Cl. C07C 7/00

(52) U.S. Cl. CPC C07C 7/005
USPC 585/802

(57) ABSTRACT

In a method for removing acid gases from hydrocarbon-comprising fluids, (a) a carbon dioxide-rich acid gas stream is separated off from the fluid by scrubbing with a liquid absorbent, (b) the fluid is contacted with a solid adsorbent for removing sulfur-comprising acid gases, and (c) the loaded solid adsorbent is regenerated by contacting with at least one purge gas under regeneration conditions. A carbon dioxide-rich acid gas stream separated off in step (a) is used as purge gas.

The present invention relates to a method for removing acid gases from hydrocarbon-comprising fluids, wherein (a) a carbon dioxide-rich acid gas stream is separated off from the fluid by scrubbing with a liquid absorbent and (b) the fluid is contacted with a solid adsorbent for removing sulfur-comprising acid gases.

Generally, it is necessary to reduce to a low concentration the acid gases such as carbon dioxide, hydrogen sulfide and organic sulfur components such as carbonyl sulfide, carbon disulfide or mercaptans, for example, that are present in hydrocarbon fluids, for example in crude natural gas, for further industrial use. A high concentration of carbon dioxide reduces the heating value of the gas and can, together with the water entrained in the gas streams, lead to corrosion on lines and reactors. Sulfur compounds present in the natural gas also form corrosive acids, together with the entrained water. In addition, numerous sulfur compounds are foul-smelling and toxic.

Frequently, scrubbing with liquid absorbents is used for removing acid gases. Suitable liquid absorbents are solutions of inorganic or organic bases such as alkali metal carbonates, primary, secondary and/or tertiary amines, or alkanolamines such as monoethanolamine (MEA), diethanolamine (DEA) and methyldiethanolamine (MDEA). On dissolution of acid gases in the absorbent, ions form with the bases. The absorbent can be regenerated by heating, expanding to a lower pressure and/or stripping, wherein the ionic species back-react to form acid gases and/or are stripped off by steam. After the regeneration process, the absorbent can be reused. Alternatively, physically acting absorbents can be used.

U.S. Pat. No. 4,336,233 discloses a method for removing CO₂ and/or H₂S from gases by scrubbing with an aqueous solution which comprises 0.05 to 0.8 mol/l of piperazine and 1.5 to 4.5 mol/l of methyldiethanolamine.

Whereas carbon dioxide can be removed to a sufficient extent by scrubbing with the liquid absorbents, the removal of sulfur-comprising acid gases poses difficulties. In particular, mercaptans, which, even at low concentration, are foul-smelling and toxic, can be removed only insufficiently by scrubbing with liquid absorbents because of the only weakly expressed acidic character of the mercaptans.

Therefore, it has already been proposed to combine the scrubbing using liquid absorbents with a treatment of the fluid using solid adsorbents such as crystalline zeolitic molecular sieves, carbon-based adsorbents, silica gels or activated aluminum oxides. When the solid adsorbent has reached a defined degree of loading with sulfur-comprising acid gases, it must be regenerated. The regeneration proceeds by temperature elevation and/or pressure reduction, in order to desorb the adsorbed sulfur-comprising acid gases again. In addition, a purge gas is passed over the solid adsorbent in order to remove the desorbed sulfur-comprising acid gases. The purge gas used is usually a substream of the previously purified fluid or a fraction thereof.

Thus U.S. Pat. No. 4,957,175 describes a method for removing carbon dioxide, hydrogen sulfide and alkylmercaptans from an inflowing gas, in which the gas is contacted with an adsorbent for removing hydrogen sulfide and alkylmercaptans, and the treated gas is contacted with a liquid absorbent for removing carbon dioxide, wherein a purified product gas is obtained. A high-boiling fraction is separated off from the purified product gas. The adsorbent that is loaded with hydrogen sulfide and alkylmercaptans is treated under regeneration conditions with a regeneration gas which is liquid under standard conditions and comprises at least some of the high-boiling fraction.

In the regeneration, a purge gas loaded with sulfur-comprising acid gases is produced which must be disposed of in a suitable manner. If the purge gas used is a substream of the fluid purified previously, or a fraction thereof, the loaded purge gas can be burnt in a combustion furnace. However, it is no longer available for adding value. In addition, increased contamination of the environment results thereby owing to carbon dioxide and sulfur oxides. Alternatively, the purge gas can be added to the crude gas for purification once more; this means an increased energy expenditure.

It is the object of the present invention to provide a more efficient and cheaper method for removing acid gases from hydrocarbon-comprising fluids. In particular, it is the object of the present invention to provide a method in which the fluid is contacted with a solid adsorbent for removing sulfur-comprising acid gases, and in which the regeneration of the solid adsorbent does not proceed, or proceeds to a minor extent, at the cost of the yield of purified fluid.

This object is achieved according to the invention by a method for removing acid gases from hydrocarbon-comprising fluids, wherein

(a) a carbon dioxide-rich acid gas stream is separated off from the fluid by scrubbing with a liquid absorbent,

(b) the fluid is contacted with a solid adsorbent for removing sulfur-comprising acid gases, and

(c) the loaded solid adsorbent is regenerated by contacting with at least one purge gas under regeneration conditions, wherein a carbon dioxide-rich acid gas stream separated off in step (a) is used as purge gas.

Hydrocarbon-comprising fluids which can be purified according to the invention are, for example, natural gas, refinery gases or reaction gases resulting from the composting of waste materials comprising organic substances, such as biogas. Natural gas is a preferred hydrocarbon-comprising fluid.

Acid gases which can be removed by the method according to the invention comprise carbon dioxide, hydrogen sulfide and organic sulfur compounds such as carbonyl sulfide, carbon disulfide or mercaptans, for example. Preferably, the fluid that is to be treated comprises at least carbon dioxide and mercaptans, in particular at least carbon dioxide, hydrogen sulfide and mercaptans, and optionally further acid gases.

The method according to the invention comprises a step (a) in which a carbon dioxide-rich acid gas stream is separated off from the hydrocarbon-comprising fluid by scrubbing with a liquid absorbent, and a step (b) in which the fluid is contacted with a solid adsorbent for removing sulfur-comprising acid gases. The steps (a) and (b) can be carried out in any desired sequence. The steps (a) and/or (b) can also be carried out repeatedly, for example using different liquid absorbents or different solid adsorbents.

In one embodiment, a carbon dioxide-rich acid gas stream is separated off from the fluid by scrubbing with a liquid absorbent, and the treated fluid is contacted with a solid adsorbent for removing sulfur-comprising acid gases. In another embodiment, the fluid is contacted with a solid adsorbent for removing sulfur-comprising acid gases and a carbon dioxide-rich acid gas stream is separated off from the treated fluid by scrubbing with a liquid absorbent.

The fluid treated by the steps (a) and (b) generally comprises less than 3% by volume, preferably less than 2% by volume, of carbon dioxide. The fluid treated by the steps (a) and (b) generally comprises less than 10 ppm by volume, preferably less than 5 ppm by volume, of hydrogen sulfide. The fluid treated by the steps (a) and (b) generally comprises less than 50 ppm by volume, preferably less than 25 ppm by volume, of mercaptans.

In step (a), a carbon dioxide-rich acid gas stream is produced. The carbon dioxide-rich acid gas stream comprises a main amount of carbon dioxide and can, in addition, comprise further acid gases such as, in particular, hydrogen sulfide. Generally, the carbon dioxide-rich acid gas stream comprises (calculated on a water-free basis) at least 85% by volume, in particular at least 95% by volume, of carbon dioxide. The exact composition of the carbon dioxide-rich acid gas stream depends on the composition of the treated fluid and on whether step (a) is carried out before or subsequently to step (b). Surprisingly, the presence of sulfur-comprising acid gases in the carbon dioxide-rich acid gas stream does not impair the suitability thereof as purge gas in the regeneration of the solid adsorbent, since the partial pressure of the sulfur-comprising acid gases in the carbon dioxide-rich acid gas stream under regeneration conditions is sufficiently low in order that under regeneration conditions effective desorption of the adsorbed sulfur-comprising acid gases proceeds.

The liquid absorbent is an absorbent which is usually used for removing carbon dioxide from fluid streams.

The liquid absorbent can be a physically acting absorbent. Absorbents are termed as such when acid gases dissolve therein substantially without changing their material properties. Principally, intermolecular interactions occur, what are termed van-der-Waals's forces. Physically acting absorbents are, for example, polypropylene carbonate, methanol, N-methylpyrrolidone, N-formylmorpholine, N-acetylmorpholine and mixtures of said substances. Suitable absorbents are, for example, known under the brand names Selexol®, Pursiol®, Genosorb® and Morphysorb®.

Preferably, however, the liquid absorbent is a chemically acting absorbent. Absorbents are termed as such which make use of a chemical reaction between the acid gases and the absorbent or one or more components of the absorbent. The chemical reaction is generally an acid-base reaction.

The chemically acting absorbent is, e.g., a solution of a base. The solvent can be aqueous or nonaqueous. Aqueous solutions are generally preferred. Nonaqueous solvents are, for example, alcohols. The base is preferably selected from alkali metal carbonates, amines, amino acid-metal salts, amidines, guanidines or combinations thereof.

Examples of nonaqueous chemically acting absorbents are mixtures of amidines and alcohols such as, e.g., 1,8-diazabicyclo-[5.4.0]-undec-7-ene (DBU) and 1-hexanol, or mixtures of guanidines and alcohols.

An aqueous solution of a base selected from alkali metal carbonates, amines, amino acid-metal salts or combinations thereof is a preferred liquid absorbent.

A suitable alkali metal carbonate is, in particular, potassium carbonate. The solution of the alkali metal carbonate can comprise activators, generally primary or secondary amines, the role of which is to accelerate the formation of the hydrogencarbonate ion by intermediate formation of a carbamate ion.

The preferred amines include alkanolamines having 2 to 12 carbon atoms. Preferred alkanolamines comprise monoethanolamine (MEA), diethanolamine (DEA), triethanolamine (TEA), diethylethanolamine (DEEA), diisopropanolamine (DIPA), aminoethoxyethanol (AEE), dimethylaminopropanol (DIMAP), methyldiethanolamine (MDEA), methyldiisopropanolamine (MDIPA) and 2-amino-1-butanol (2-AB).

Particularly preferred absorbents comprise at least one tertiary alkanolamine and preferably one activator in the form of a primary or secondary amine. Preferred activators are saturated, 5- to 7-membered heterocyclic compounds having at least one NH group and optionally a further heteroatom in the ring selected from an oxygen and a nitrogen atom. Suitable activators are piperazine, 1-methylpiperazine, 2-methyl-piperazine, 1-aminoethylpiperazine, morpholine, piperidine. Other preferred activators are selected from methylaminopropylamine, 2-amino-1-butanol and aminoethoxyethanol.

The absorbent described in U.S. Pat. No. 4,336,233 has also proven particularly useful. This is an aqueous solution of methyldiethanolamine (MDEA) and piperazine as activator.

If aqueous solutions of amines are used as liquid absorbent, they preferably have a total amine content of 30 to 70% by weight, in particular 35 to 50% by weight.

Suitable amino acid-metal salts are, for example, N,N-dimethylaminoacetic acid-potassium salt, N,N-diethylaminoacetic acid-potassium salt and N-ethyl-N-methylaminoacetic acid-potassium salt.

Mixtures of physically acting absorbents and aqueous solutions of a base can also be used, for example a mixture of water, sulfolane and either DIPA or MDEA.

Generally, the fluid is contacted with the liquid absorbent in step (a), wherein a loaded liquid absorbent is obtained, and the loaded liquid absorbent is regenerated by heating, expanding and/or stripping, wherein the carbon dioxide-rich acid gas stream is obtained. The regenerated liquid absorbent is used again for scrubbing the fluid.

The fluid is contacted with the liquid absorbent in a suitable absorber. Suitable absorbers are, for example, random-packing, arranged-packing and tray columns. In tray columns, sieve trays, bubble-cap trays or valve trays are installed, over which the liquid flows. The vapor is passed through special slots or holes in such a manner that a spouted bed forms. On each of these trays, a new equilibrium is formed. Random-packing columns can be packed with different shaped bodies. Heat exchange and mass transfer are improved via the increase in surface area owing to the shaped bodies that are usually about 25 to 80 mm in size. Known examples are the Raschig Ring (a hollow cylinder), Pall Ring, Hiflow Ring, Intalox Saddle and the like. The packings can be introduced into the column in an arranged, but also random (as bed), manner. Materials which come into consideration are glass, ceramic, metal and plastics. Structured packings are a further development of the arranged packings. They have a regularly shaped structure. It is possible thereby with packings to reduce pressure drops in the gas flow. There are various designs of packings, e.g. fabric or sheet metal packings. Metal, plastic, glass and ceramic can be used as material.

The temperature of the absorption is between about 40° C. and about 100° C. at a pressure between about 1 bar and about 120 bar.

The liquid absorbent is preferably regenerated using pressure expansion and stripping in a desorption column. The desorption column can likewise be a random-packing, arranged-packing or tray column. The desorption column has a reboiler at the bottom, e.g. a forced circulation evaporator having a circulation pump. The loaded liquid absorbent is generally introduced into the desorption column in the upper region, whereas a stripping medium flows in the opposite direction from the bottom. The stripping medium is generally a hot gas or steam, preferably steam.

At the top the desorption column has an outlet for the liberated acid gases. Entrained absorbent vapors are condensed in a condenser and recirculated to the column. The regenerated liquid absorbent can be reused for removing carbon dioxide from the fluid.

The liquid absorbent is generally regenerated at a temperature of about 35° C. to about 150° C., preferably from about 90° C. to about 130° C., and a pressure from about 1 bar to about 5 bar, preferably from about 1 bar to about 3 bar.

In section (b), the fluid is contacted in an adsorbent contact zone with a solid adsorbent for removing sulfur-comprising acid gases. The sulfur-comprising acid gases removed in this step preferably comprise at least mercaptans, usually hydrogen sulfide and mercaptans.

Suitable solid adsorbents are zeolites which are also termed molecular sieves, carbon-based adsorbents, silica gels or activated aluminum oxides.

Preferably, zeolites having a pore size of at least about 4.6 Å are used. Suitable zeolites are, for example, zeolite A (LTA), zeolite X or Y (FAU faujasite family) or zeolite MFI (ZSM-5 and silicalite). Specific examples are zeolite 5A, zeolite 13X, zeolite 4A or mixtures thereof.

Among the A zeolites (LTA), zeolite 4A is particularly suitable, the sodium ions of which are preferably partially replaced by calcium. The Na/Ca degree of exchange is preferably 25 to 85 mol %. Among the zeolites of type X or Y (faujasite FAU), zeolite 13X (NaX) is particularly suitable, wherein other cations, for instance Ca, Ba, Li, Sr, Mg, Rb, Cs, Cu, Ag, can be used for charge balancing. The Si/Al molar ratio can be from 1 to infinity (such as in dealuminized Y zeolites, for example). In these the ratio is infinite. Among the MFI zeolites, ZSM-5 are suitable having an Si/AI molar ratio from 1 to infinity (such as in silicalite, for example).

The carbon-based adsorbents include activated carbons, preferably those having a BET surface area (determined by physisorption of nitrogen at 77 K) from 200 to 2000 m²/g.

In addition, silica gels or activated aluminum oxides are suitable, preferably those having a BET surface area from 100 to 800 m²/g.

The solid adsorbent is preferably in particulate form, for example as beads or extruded rods. The adsorbents, before use thereof in the method according to the invention, can have been subjected to a usual shaping method such as, for example, pelletizing, tableting or extrusion. The median particle size is, for example, 2 to 25 mm. The solid adsorbent is preferably used in the form of a bed or as a fixed bed.

The fluid is generally contacted with the adsorbent by passing the fluid over a bed of the adsorbent. The linear velocity of the fluid in this case is preferably in a range from about 1 to 200 cm/min. Suitable, optionally pressure-resistant, reaction apparatuses are known to those skilled in the art for contacting the fluid with the above described adsorbents. These include the generally conventional reactors for gas/solid reactions and liquid/solid reactions. Preferably, a vertical, elongate fixed-bed reactor is used, through which the fluid that is to be purified flows in the direction of gravity or in the opposite direction to gravity. If desired, the adsorbent can be used in the form of a single contact zone or in the form of a plurality of contact zones. In this case, each of these zones can comprise one or more of the above described adsorbents.

Preferably, the fluid is contacted with the adsorbent at a temperature in the range from about 0 to 70° C., preferably 5 to 60° C., in particular 10 to 50° C.

Preferably, the fluid is contacted with the adsorbent at a pressure in the range of about 1 to 200 bar, preferably about 1 to 100 bar, in particular about 40 to 100 bar, and especially about 40 to 80 bar.

By contacting the fluid with the solid adsorbent, sulfur-comprising acid gases, in particular mercaptans, are adsorbed to the adsorbent and in this manner removed from the fluid. When the solid adsorbent has reached a defined degree of loading with sulfur-comprising acid gases, it is regenerated. For this purpose the loaded solid adsorbent is contacted with at least one purge gas under regeneration conditions. According to the invention, a carbon dioxide-rich acid gas stream separated off in step (a) is used as purge gas. This means that at least a part of the carbon dioxide-rich acid gas stream separated off in step (a) is used as purge gas. The regenerated solid adsorbent is then used at least in part again in step (b).

In addition to the carbon dioxide-rich acid gas stream, other purge gases can also be used for regeneration of the loaded solid adsorbent, for example before or subsequently to the regeneration with the carbon dioxide-rich acid gas stream. For example, a coarse regeneration of the loaded solid adsorbent can proceed using the carbon dioxide-rich acid gas stream separated off in step (a) and a fine regeneration using at least one further gas. The further gas can be a fluid treated by the steps (a) and (b), or a fraction thereof, e.g. a high- or medium-boiling fraction of the treated fluid.

The regeneration proceeds under regeneration conditions, i.e. conditions of temperature and pressure which permit a desorption of sulfur-comprising acid gases from the loaded solid adsorbent.

Generally, the regeneration in step (c) is carried out at a regeneration temperature which is higher than the temperature of step (b). The regeneration of the adsorbent generally proceeds at a regeneration temperature of 50° C. to 370° C., preferably 50° C. to 320° C.; the regeneration pressure is generally 1 to 50 bar, preferably 1 to 10 bar.

In order to set the regeneration temperature, preferably the purge gas used for regenerating the loaded solid adsorbent is preheated before it is contacted with the loaded solid adsorbent. Alternatively, the bed of the adsorbent can be actively heated, but this is less preferred. For energy integration, the influent purge gas can be preheated by indirect heat exchange with the purge gas flowing away from the solid adsorbent (and loaded with the desorbed sulfur-comprising acid gases).

Preferably, the regenerated solid adsorbent is cooled from the regeneration temperature to the temperature of step (b) by contact with a cooling gas, before it is again contacted with the fluid that is to be treated. The cooling gas can be fluid treated by the steps (a) and (b), a fraction thereof, or a carbon dioxide-rich acid gas stream separated off in step (a), for example.

In a preferred embodiment, at least two adsorbent contact zones are provided, through which the fluid that is to be treated can flow alternately. In at least one first adsorbent contact zone, the fluid that is to be treated is contacted with the solid adsorbent, whereas the loaded solid adsorbent is regenerated and/or cooled in at least one further zone.

After regeneration is completed, the fluid that is to be treated can be diverted and guided through the further adsorbent contact zone, while the loaded solid adsorbent is regenerated in the first zone.

Hereinafter the invention will be described in more detail with reference to working examples shown in the drawings. In the drawings:

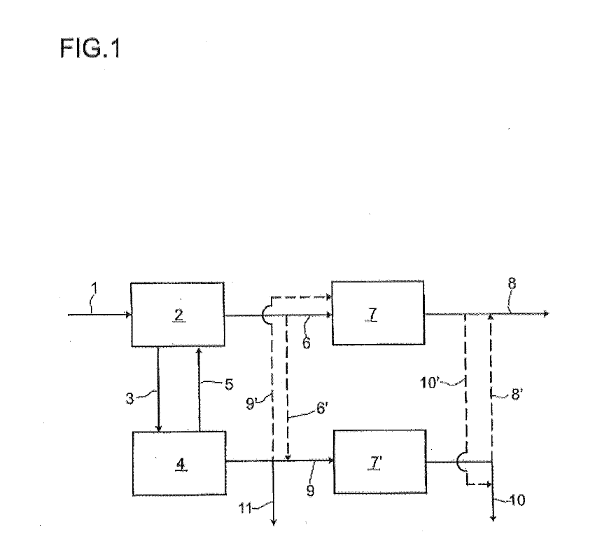

FIG. 1 shows a schematic depiction of a plant suitable for carrying out the method according to the invention,

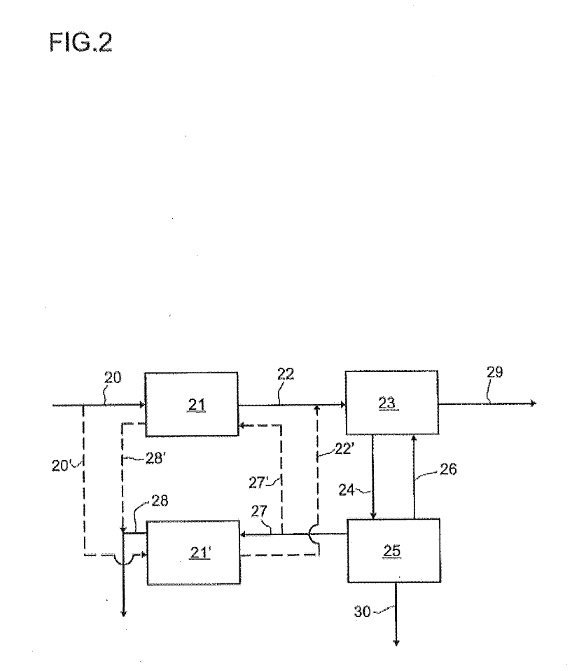

FIG. 2 shows a schematic depiction of another plant suitable for carrying out the method according to the invention.

FIG. 1 is a schematic depiction of a plant suitable for carrying out the method according to the invention. The plant comprises an absorption column 2, a desorption column 4 and also two adsorbent contact zones (7 and 7′). The carbon dioxide is removed in this case from the fluid stream before removal of the mercaptans. FIG. 1 shows the two possible circuits of the plant, wherein the one circuit is indicated by continuous lines, and the other by means of dashed lines. Via a feed line 1, a fluid rich in acid gases is passed into the absorption column 2 where it is scrubbed in counter flow with the liquid absorbent 5. In this process a fluid stream 6 that is depleted in carbon dioxide is obtained and also an aqueous absorbent loaded with carbon dioxide. The carbon dioxide-loaded liquid absorbent is passed via line 3 into the upper part of the desorption column 4 and, under reduced pressure and/or increased temperature, is freed from the absorbed carbon dioxide and optionally other acid gases. The aqueous absorbent thus regenerated can be passed via line 5 back into the upper part of the absorption column 2.

The fluid stream that is depleted in carbon dioxide is passed via line 6 into one of the two adsorbent contact zones 7. In the adsorbent contact zone 7, sulfur-comprising acid gases are removed from the fluid by adsorption to the solid adsorbent. The fluid thus freed from acid gases leaves the plant via line 8.

The carbon dioxide-rich acid gas stream occurring in the desorption column 4 is in part ejected via line 11 and in part passed via line 9 into the other of the two adsorbent contact zones 7′. In the adsorbent contact zone 7′, in this case the regeneration of the loaded solid adsorbent proceeds via contact with the carbon dioxide-rich acid gas stream. The adsorbent contact zone 7′ can pass through various stages of the adsorbent regeneration. The regeneration comprises heating the loaded adsorbent via the previously heated carbon dioxide-rich acid gas stream that is fed through line 9 (heat exchanger not shown) and the subsequent cooling by a suitable medium of the solid adsorbent thus regenerated. The carbon dioxide-rich acid gas stream that is loaded with the desorbed sulfur-comprising acid gases is removed via line 10 and disposed of in a suitable manner.

After a time period, the plant is switched over and the fluid stream that is depleted in carbon dioxide is passed via line 6′ into the other of the two adsorbent contact zones 7′. Then, in the adsorbent contact zone 7′, sulfur-comprising acid gases are removed from the fluid by adsorption to the solid adsorbent. The fluid thus freed from acid gases leaves the plant via line 8′ and 8. In the adsorbent contact zone 7, the loaded solid adsorbent is then regenerated by contact with the carbon dioxide-rich acid gas stream which is fed via the line 9′. The carbon dioxide-rich acid gas stream loaded with the desorbed sulfur-comprising acid gases is removed via line 10′ and 10 and disposed of in a suitable manner.

FIG. 2 is a schematic depiction of another plant suitable for carrying out the method according to the invention. The plant comprises two adsorbent contact zones (21 and 21′) and also an absorption column 23 and a desorption column 25. The carbon dioxide is removed from the fluid stream in this case after the removal of the mercaptans. FIG. 2 shows the two possible circuits of the plant, wherein the one circuit is indicated by continuous lines, and the other by dashed lines. Via a feed line 20, an acid gas-rich fluid is passed into one of the two adsorbent contact zones 21. In the adsorbent contact zone 21, sulfur-comprising acid gases are removed from the fluid by adsorption to the solid adsorbent. The fluid thus freed from sulfur-comprising acid gases is passed via line 22 into the bottom part of the absorption column 23, where it is scrubbed with liquid absorbent 26 in counter flow. In this case a product fluid stream that is depleted in acid gases and a carbon dioxide-loaded liquid absorbent are obtained. The product fluid that is depleted in acid gases leaves the plant via line 29.

The liquid absorbent loaded with carbon dioxide is passed via line 24 into the upper part of the desorption column 25 and freed from the absorbed carbon dioxide and optionally further acid gases under reduced pressure and/or increased temperature. The liquid absorbent thus regenerated is passed via line 26 back into the upper part of the absorption column 23. The carbon dioxide-rich acid gas stream that is separated off is in part ejected via line 30 and in part passed via line 27 into the adsorbent contact zone 21′. In the adsorbent contact zone 21′, the regeneration of loaded solid adsorbent proceeds via contact with the carbon dioxide-rich acid gas stream which is supplied via line 27. The carbon dioxide-rich acid gas stream that is loaded with the desorbed sulfur-comprising acid gases is removed via line 28 and disposed of in a suitable manner.

After a time period, the plant is switched over and the fluid rich in acid gases is passed via line 20′ into the other of the two adsorbent contact zones 21′. Then, in the adsorbent contact zone 21′, sulfur-comprising acid gases are removed from the fluid by adsorption to the solid adsorbent. The fluid thus freed from sulfur-comprising acid gases is passed via line 22′ into the lower part of the absorption column 23. In the adsorbent contact zone 21, then the regeneration of the loaded solid adsorbent proceeds by contact with the carbon dioxide-rich acid gas stream which is fed via the line 27′. The carbon dioxide-rich acid gas stream that is loaded with the desorbed sulfur-comprising acid gases is removed via line 28′ and 28 and disposed of in a suitable manner.

1.-14. (canceled)

15. A method for removing acid gases from hydrocarbon-comprising fluids, wherein
(a) a carbon dioxide-rich acid gas stream is separated off from the fluid by scrubbing with a liquid absorbent,
(b) the fluid is contacted with a solid adsorbent for removing sulfur-comprising acid gases, and
(c) the loaded solid adsorbent is regenerated by contacting with at least one purge gas under regeneration conditions, wherein a carbon dioxide-rich acid gas stream separated off in step (a) is used as purge gas.

16. The method according to claim 15, wherein the liquid absorbent is a physically acting absorbent.

17. The method according to claim 15, wherein the liquid absorbent is a chemically acting absorbent.

18. The method according to claim 17, wherein the liquid absorbent is a solution of a base.

19. The method according to claim 18, wherein the liquid absorbent is an aqueous solution of a base selected from alkali metal carbonates, amines, amino acid-metal salts or combinations thereof.

20. The method according to claim 15, wherein the solid adsorbent is selected from zeolites, carbon-based adsorbents, silica gels, activated aluminum oxides and combinations thereof.

21. The method according to claim 20, wherein the zeolites have a pore size of at least about 4.6 Å.

22. The method according to claim 15, wherein at least one further gas different from the carbon dioxide-rich acid gas stream is used as purge gas.

23. The method according to claim 22, wherein the further gas is a fluid treated by the steps (a) and (b), or a fraction thereof.

24. The method according to claim 15, wherein the regeneration conditions comprise a regeneration temperature which is above the temperature of step (b).

25. The method according to claim 24, wherein the regeneration temperature is 50° C. to 370° C.

26. The method according to claim 24, wherein the regenerated solid adsorbent is cooled from the regeneration temperature to the temperature of step (b) by contact with a cooling gas.

27. The method according to claim 26, wherein the cooling gas is fluid treated by the steps (a) and (b), a fraction thereof, or a carbon dioxide-rich acid gas stream separated off in step (a).

28. The method according to claim 15, wherein the fluid is contacted with the liquid absorbent in step (a), wherein a loaded liquid absorbent is obtained, and the loaded liquid absorbent is regenerated by heating, expanding and/or stripping, wherein the carbon dioxide-rich acid gas stream is obtained.
